(19) United States
(12) Patent Application Publication
(10) Pub. No.: US 2007/0093857 A1
(43) Pub. Date: Apr. 26, 2007

(54) Devices and methods for treating mitral valve regurgitation

(76) Inventors: Campbell Rogers, Dedham, MA (US); Adam Groothuis, Swampscott, MA (US)

Correspondence Address: EDWARDS & ANGELL, LLP, P.O. BOX 55874, BOSTON, MA, 02205, US

(21) Appl. No.: 11/586,201

(22) Filed: Oct. 25, 2006

Related U.S. Application Data
(60) Provisional application No. 60/730,410, filed on Oct. 26, 2005.

(51) Int. Cl. A61B 17/10

(52) U.S. Cl. 606/142

(57) ABSTRACT

A medical device, method and system of treating the lumenal system of a patient are provided. The medical device includes a tissue plicator adapted and configured to form a plication of tissue proximate a target region of a patient. The medical device further includes a retainer applicator operatively associated with the tissue plicator. The retainer applicator is adapted and configured to apply a retainer to the plication to maintain the plication after the medical device is removed from the patient. In accordance with a further aspect, the tissue plicator may plicate tissue by mechanically clamping the tissue and/or may plicate the tissue at least in part by applying suction thereto. The system can be used to plicate tissue proximate the mitral valve of a patient. The plication can be formed temporarily or permanently.

### CROSS-REFERENCE TO RELATED APPLICATION

This application claims the benefit of priority from U.S. Provisional Patent Application Ser. No. 60/730,410, filed Oct. 26, 2005, the disclosure of which is incorporated by reference herein in its entirety.

### BACKGROUND OF THE INVENTION

1. Field of the Invention

The present invention relates to a method and system for treating the luminal system of a patient. Particularly, the present invention is directed to a method and system for treating mitral valve regurgitation.

2. Description of Related Art

Mitral regurgitation, or leakage, is the backflow of blood from the left ventricle into the left atrium due to an imperfect closure of the mitral valve. Leakage often occurs when a gap is created between the anterior leaflet and posterior leaflet of the mitral valve. A variety of methods and systems are known in the art for treating mitral valve regurgitation. Of such devices, many are directed to open surgical techniques as well as complex endoscopic techniques that can be difficult to perform.

In general, a relatively significant gap may exist between the anterior leaflet and posterior leaflet of the mitral valve for a variety of different reasons. For example, a gap may exist due to congenital malformations, because of ischemic disease, or because a heart has been damaged by a previous heart attack. A gap may also be created when congestive heart failure, e.g., cardiomyopathy, or some other type of distress causes a heart to be enlarged. When a heart is enlarged, the walls of the heart, e.g., wall of a left ventricle, may stretch or dilate, causing the posterior leaflet of the mitral valve to stretch. It should be appreciated that anterior leaflet of the mitral valve generally does not stretch. Accordingly, a gap can be created between the leaflets of the mitral valve when the walls of the left ventricle stretch. Hence, due to the existence of the gap, the mitral valve is unable to close properly, and may begin to leak. Leakage through the mitral valve generally causes a heart to operate less efficiently, as the heart must work harder to maintain a proper amount of blood flow therethrough.

Treatments used to correct for mitral valve leakage are typically highly invasive, open-heart surgical procedures. Ventricular assist devices such as artificial hearts may be implanted in a patient whose own heart is failing. The implantation of a ventricular assist device is often expensive, and a patient with a ventricular assist device must be placed on extended anti-coagulant therapy. As will be appreciated by those skilled in the art, anti-coagulant therapy reduces the risk of blood clots being formed, as for example, within the ventricular assist device. While reducing the risks of blood clots associated with the ventricular assist device is desirable, anti-coagulant therapies may increase the risk of uncontrollable bleeding in a patient, e.g., as a result of a fall, which is not desirable.

Open-heart surgical procedures which are intended to correct for mitral valve leakage, specifically, involve the implantation of replacement valves. Valves from animals, e.g., pigs, may be used to replace a mitral valve in a human. While the use of a pig valve may relatively successfully replace a mitral valve, such valves generally wear out, thereby requiring additional open surgery at a later date. Mechanical valves, which are less likely to wear out, may also be used to replace a leaking mitral valve. However, when a mechanical valve is implanted, there is an increased risk of thromboembolism, and a patient is generally required to undergo extended anti-coagulant therapies.

One open-heart surgical procedure that is particularly successful in correcting for mitral valve leakage and, in addition, mitral regurgitation, is an annuloplasty procedure. During an annuloplasty procedure, an annuloplasty ring may be implanted on the mitral valve to cause the size of a stretched mitral valve to be reduced to a relatively normal size. An annuloplasty ring is shaped approximately like the contour of a normal mitral valve. That is, an annuloplasty ring is shaped substantially like the letter “D.” Typically, annuloplasty rings may be formed from a rod or tube of biocompatible material, e.g., plastic, that has a DACRON mesh covering.

In order for an annuloplasty ring to be implanted, a surgeon surgically attaches the annuloplasty ring to the mitral valve on the atrial side of the mitral valve. Conventional methods for installing such a ring require open-heart surgery which involve opening a patient's sternum and placing the patient on a heart bypass machine. The annuloplasty ring is sewn to the posterior leaflet and the anterior leaflet of a top portion of the mitral valve. In sewing the annuloplasty ring onto the mitral valve, a surgeon generally alternately acquires a relatively large amount of tissue from mitral tissue, e.g., a one-eighth inch bite of tissue, using a needle and thread, followed by a smaller bite from the annuloplasty ring. Once a thread has loosely coupled the annuloplasty ring to the mitral valve tissue, the annuloplasty ring is slid onto the mitral valve such that tissue that was previously stretched out, e.g., due to an enlarged heart, is effectively pulled in using tension applied by the annuloplasty ring and the thread which binds the annuloplasty ring to the mitral valve tissue. As a result, the gap between the anterior leaflet and the posterior leaflet may be substantially closed off. After the mitral valve is shaped by the annuloplasty ring, the anterior and posterior leaflets of the mitral valve will reform to create a new contact line and will enable the mitral valve to appear and to function as a normal mitral valve.

Once implanted, tissue generally grows over the annuloplasty ring, and a line of contact between the annuloplasty ring and the mitral valve will essentially enable the mitral valve to appear and function as a normal mitral valve. Although a patient who receives the annuloplasty ring may be subjected to anti-coagulant therapies, the therapies are not extensive, as a patient is only subjected to the therapies for a matter of weeks, e.g., until tissue grows over the annuloplasty ring.

A second surgical procedure which is generally effective in reducing mitral valve leakage involves placing an edge-to-edge suture in the mitral valve. Such a surgical procedure, e.g., an Alfieri stitch procedure or a bow-tie repair procedure, will be described. An edge-to-edge stitch is used to stitch together an area at approximately the center of a gap defined between the anterior and posterior leaflets of the mitral valve. Once the stitch is in place, the stitch is pulled in to form a suture which holds anterior leaflet against the posterior leaflet, as shown. By reducing the size of the gap between the anterior leaflet and the posterior leaflet, the amount of leakage through the mitral valve may be substantially reduced.

Although the placement of an edge-to-edge stitch is generally successful in reducing the amount of mitral valve leakage through the gap between the leaflets of the mitral valve, this technique is conventionally made through open-heart surgery. In addition, the use of the edge-to-edge stitch is generally not suitable for a patient with an enlarged, dilated heart, as blood pressure causes the heart to dilate outward, and may put a relatively large amount of stress on the edge-to-edge stitch.

While invasive surgical procedures have proven to be effective in the treatment of mitral valve leakage, invasive surgical procedures often have significant drawbacks. Any time a patient undergoes open-heart surgery, there is a risk of infection. Opening the sternum and using a cardiopulmonary bypass machine has also been shown to result in a significant incidence of both short and long term neurological deficits.

Thus, there still remains a continued need in the art for a minimally invasive technique for treating mitral valve regurgitation that permits a surgeon greater control over tuning the performance of the mitral valve and that minimizes trauma to the patient. The present invention provides a solution for these problems, as described herein.

### SUMMARY OF THE INVENTION

The purpose and advantages of the present invention will be set forth in and apparent from the description that follows, as well as will be learned by practice of the invention. Additional advantages of the invention will be realized and attained by the methods and systems particularly pointed out in the written description and claims hereof, as well as from the appended drawings.

To achieve these and other advantages and in accordance with the purpose of the invention, as embodied herein and broadly described, the invention includes a medical device. The medical device includes a tissue plicator adapted and configured to form a plication of tissue proximate a target region of a patient. The medical device further includes a retainer applicator operatively associated with the tissue plicator. The retainer applicator is adapted and configured to apply a retainer to the plication to maintain the plication after the medical device is removed from the patient.

In accordance with a further aspect, the tissue plicator may plicate tissue by mechanically clamping the tissue. The tissue plicator may include forceps adapted to mechanically grasp the tissue. If desired, the forceps may include a plurality of teeth for gripping tissue. Additionally or alternatively, the tissue plicator may plicate the tissue at least in part by applying suction thereto. In accordance with this aspect, a sheath can be provided defining a lumen. The tissue plicator may plicate the tissue by drawing the tissue into the lumen. A mechanical plicator can be disposed in the lumen to assist plication, if desired. The mechanical plicator can be adapted to expand the sheath in a radial direction when grasping tissue. The entirety or portions of the tissue plicator may be formed at least in part of radiopaque material, among other materials.

In accordance with another aspect of the invention, the retainer applicator can be adapted and configured to deliver the retainer along the tissue plicator to the target region. In accordance with this aspect, the retainer applicator can adapted and configured to deliver the retainer along the outside of the tissue plicator to the target region. Alternatively, if desired, the tissue plicator can define a lumen therethrough and the retainer applicator can be adapted and configured to deliver the retainer along the lumen defined by the tissue plicator to the target region.

In accordance with still another aspect, a plicator actuator can be provided operably coupled to the tissue plicator. The plicator actuator is configured and adapted to adjust the tissue plicator from a first configuration wherein the tissue plicator is disengaged from the target area to an second configuration wherein the tissue plicator is engaged with the target area. Additionally or alternatively, an applicator actuator can be operably coupled to the retainer applicator, wherein the actuator is configured and adapted to affix the retainer onto the plication of tissue.

In accordance with another aspect of the invention, a medical device is provided having a tissue plicator adapted and configured to form a plication of tissue in endocardial muscular tissue proximate the mitral valve of a patient. The medical device can include a retainer applicator operatively associated with the tissue plicator, wherein the retainer applicator is adapted and configured to apply a retainer to the plication to maintain the plication after the medical device is removed from the patient.

In accordance with still another aspect of the invention, a medical device is provided having a retainer applicator adapted and configured to apply a retainer to a plication of tissue by rotating the retainer about a longitudinal axis defined by the retainer applicator to maintain the plication after the medical device is removed from the patient.

In further accordance with the invention, a system is provided. The system includes a inner catheter as described herein. The system further includes a retainer configured and adapted to maintain the plication of tissue after the device is removed from the patient.

In accordance with a further aspect of the system, an outer catheter can also be provided defining a first lumen, wherein the inner catheter is disposed in the first lumen. The outer catheter can further define a second lumen parallel to the first lumen. The second lumen can be connected to a source of beneficial agent, and the system can be adapted and configured to selectively deliver the beneficial agent to the target region. The beneficial agent can be chosen from the group consisting of contrast agents, medicaments, viral vectors, and genetic material, among others. Moreover, a stiffening wire can be disposed in the second lumen. The stiffening wire can be movably disposed in the second lumen or can be stationary, if desired. The stiffening wire can have a varying stiffness along its length.

In accordance with still a further aspect of the system, the retainer can include a main body portion having a proximal end and a distal end, wherein the proximal end of the main body portion can define a mating portion for mating with the applicator. The retainer can be further provided with a distal end including a first prong adapted and configured to pass through tissue of a patient's vascular system. The retainer can include a second prong attached to the main body portion. The second prong can be deformable from an open position for capturing a tissue plication between the first prong and second prong to a closed position for maintaining a tissue plication by the applicator. The mating portion of the retainer can define a loop adapted and configured to receive a portion of the applicator. Moreover, the retainer can include a third prong attached to the main body portion, wherein the second prong and third prong are generally parallel to the main body portion in the open position.

In accordance with another embodiment, the retainer can be substantially ring shaped. In accordance with this aspect, the retainer can be deformed from an open position to a closed position for capturing and maintaining a tissue plication. If desired, the retainer can adapted and configured to be folded by the applicator about the tissue plication. Additionally or alternatively, the retainer can be helically shaped and rotated about a longitudinal axis defined by the medical device to introduce the retainer into the target region. The retainer can be made from a variety of materials, including, for example, shape memory materials, radiopaque materials, resorbable materials, polymeric materials, echogenic materials and/or fluoroscopically visible materials. The retainer can also include one or more barbs for anchoring the retainer into the patient.

In further accordance with the invention, a method is provided. The method includes the steps of providing a inner catheter having a distal portion for creating a plication in tissue, introducing the inner catheter into a lumenal system of a patient, and advancing the distal portion to a target region to be plicated. The method further includes the steps of temporarily plicating tissue proximate the target region to form a first plication, applying a first retainer to the first plication and removing the inner catheter from the patient.

In further accordance with the invention, the target region can be proximate the mitral valve of a patient. The first plication can be formed, for example, on the ventricular wall or the atrial wall. The target region can be proximate the posterior leaflet of the mitral valve. The retainer may be introduced into the patient by sliding it over the inner catheter.

In accordance with another aspect, the method can further include the step of observing the circulation through the patient's heart after plicating the tissue but before applying the retainer to determine if mitral regurgitation has been decreased by plicating the tissue. If desired, the plication can be released and a new plication can be formed in order to improve the regurgitation of the mitral valve. To assist in this procedure, the circulation of the patient can be observed, for example by using a fluoroscopic technique or ultrasonographic technique, among others. The inner catheter can be introduced into a patient through a lumen of an outer catheter.

In accordance with another aspect, the mitral valve of the patient can define a perimeter and a plication can be formed in the cardiac tissue to reduce the perimeter of the mitral valve. Additional plications proximate the mitral valve and radially displaced from the first plication can be formed, if desired. These plications can be held in place by additional retainers to further reduce the perimeter of the mitral valve.

In accordance with yet another aspect, a method is provided including the steps of providing a inner catheter having a distal portion for creating a plication in tissue, introducing the inner catheter into a lumenal system of a patient, advancing the distal portion proximate an endocardial location and engaging endocardial tissue to form a plication therein. The endocardial tissue can be proximate a mitral valve of a patient for purposes of treating mitral valve regurgitation, for example. In accordance with one embodiment, the endocardial tissue is muscular tissue. The plication can be formed temporarily by a device such as a tissue forceps and/or can be formed by applying a retainer to the endocardial tissue. The plication can be formed on the ventricular and/or atrial walls.

The invention provides still another alternate method, including the steps of providing a inner catheter having a distal portion for creating a plication in tissue, introducing the inner catheter into a lumenal system of a patient, advancing the distal portion proximate an interior surface of the lumenal system, engaging endocardial tissue to form a plication therein and attaching a retainer to maintain the plication by rotating the retainer about a longitudinal axis defined by the inner catheter.

It is to be understood that both the foregoing general description and the following detailed description are exemplary and are intended to provide further explanation of the invention claimed.

The accompanying drawings, which are incorporated in and constitute part of this specification, are included to illustrate and provide a further understanding of the method and system of the invention. Together with the description, the drawings serve to explain the principles of the invention.

### BRIEF DESCRIPTION OF THE DRAWINGS

FIGS. 1(a)-1(e) are schematic views of portions of first and second representative embodiments of a system in accordance with the present invention.

FIG. 2 is a schematic view of a portion of a third representative embodiment of a system in accordance with the present invention.

FIG. 3 is a schematic view of a portion of a fourth representative embodiment of a system in accordance with the present invention.

FIG. 4 is a schematic view of a portion of the embodiment of FIG. 1(a).

FIG. 5 is a schematic view of a portion of a fifth representative embodiment of a system in accordance with the present invention.

FIG. 6 is a cross-sectional view of a portion of the embodiment of FIG. 1(a).

FIGS. 7(a)-7(e) are schematic views of different embodiments of retainers made in accordance with the present invention.

FIGS. 8(a)-8(b) are partial schematic views of a sixth representative embodiment of a system made in accordance with the present invention.

FIG. 9 is a schematic representation illustrating a method in accordance with the present invention.

FIGS. 10(a)-10(b) are schematic representations illustrating a method in accordance with the present invention.

### DETAILED DESCRIPTION OF THE PREFERRED EMBODIMENT

Reference will now be made in detail to the present preferred embodiments of the invention, an example of which is illustrated in the accompanying drawings. The method and corresponding steps of the invention will be described in conjunction with the detailed description of the system.

The devices and methods presented herein may be used for treating the luminal system of a patient. The present invention is particularly well suited for treating valve regurgitation, such as mitral valve regurgitation. In accordance with the invention, a medical device is provided including a tissue plicator adapted and configured to form a plication of tissue proximate a target region of a patient.

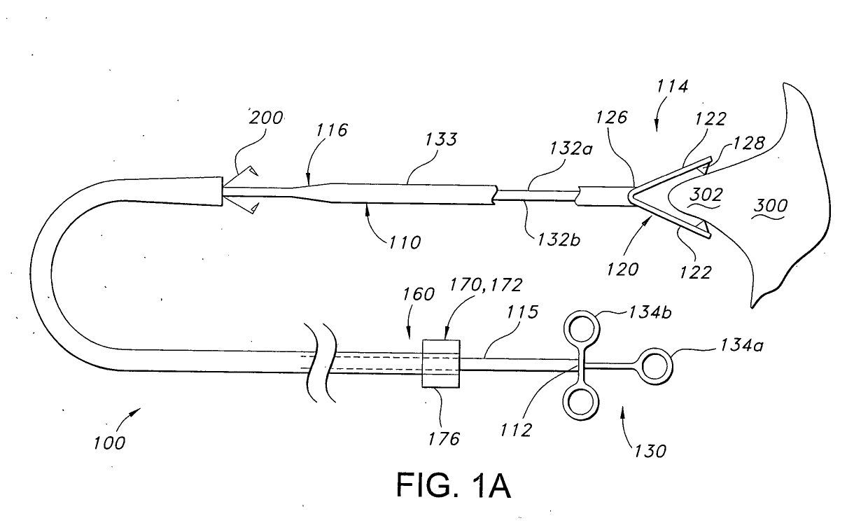

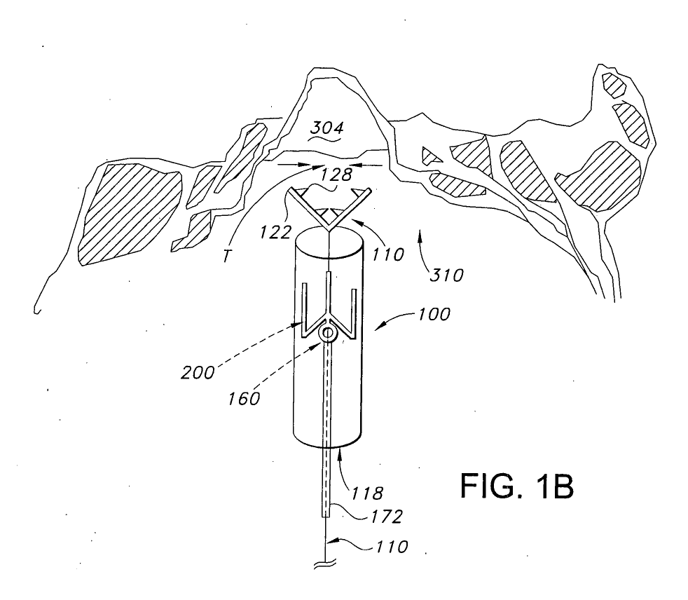

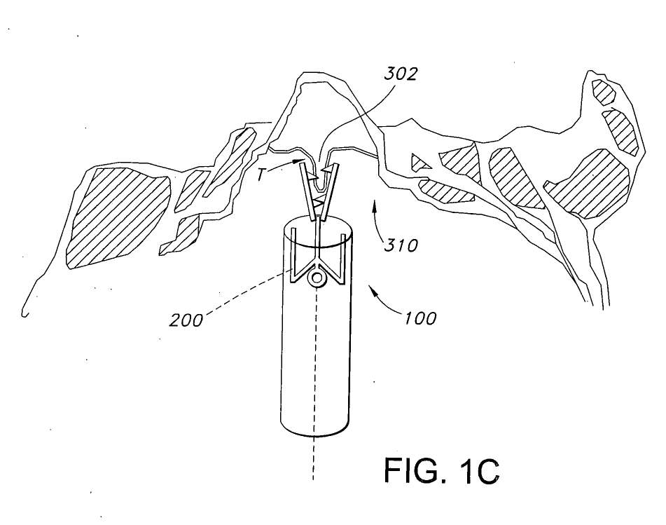

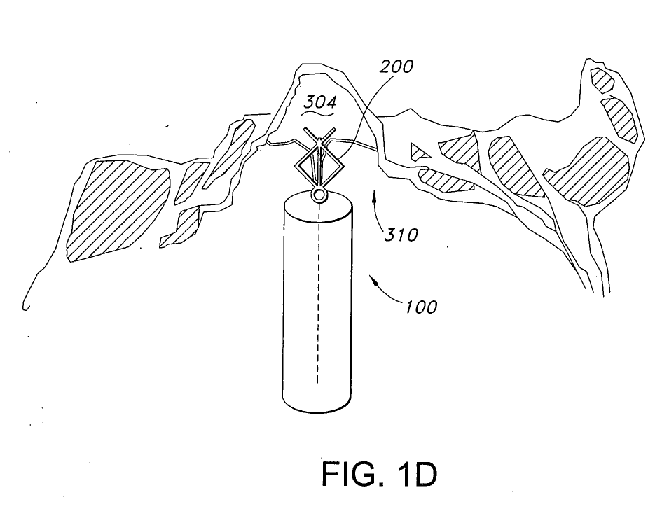

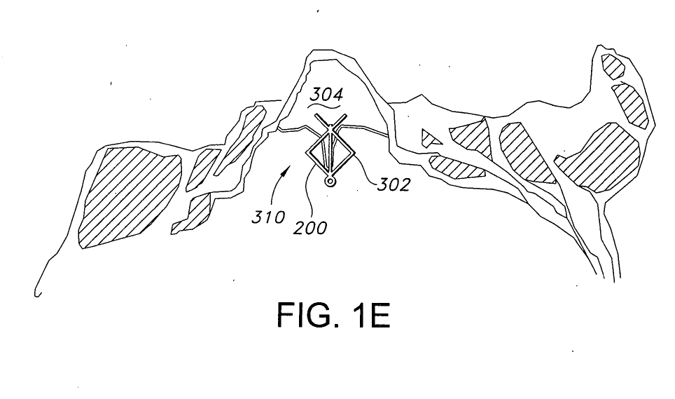

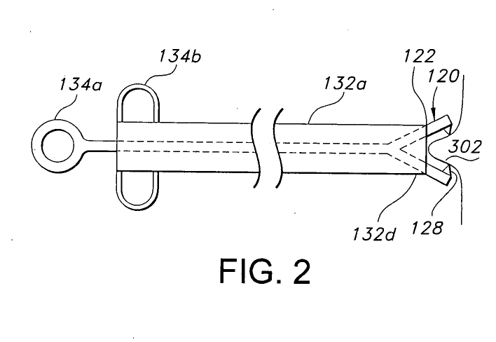

For purpose of explanation and illustration, and not limitation, a partial view of an exemplary embodiment of the medical device in accordance with the invention is shown in FIG. 1(a) and is designated generally by reference character 100. Other embodiments of a medical device in accordance with the invention, or aspects thereof, are provided in FIGS. 2-10, as will be described.

In accordance with the invention, the medical device includes a tissue plicator adapted and configured to form a plication of tissue proximate a target region of a patient.

For purposes of illustration and not limitation, as embodied herein and as depicted in FIG. 1(a), medical device 100 is provided with a tissue plicator 110. As depicted in FIG. 1(a), tissue plicator 110 includes a proximal end 112, a distal end 114 and includes an elongate body 116. In the embodiment of FIG. 1(a), tissue plicator plicates tissue 300 by mechanically clamping tissue 300 using forceps 120. Forceps 120 include first and second jaws 122 that are adapted to open and close about a hinge 126 to mechanically grasp tissue 300 to form a plication 302. Hinge 126 can be an actual hinge with a pivot, or can be a living hinge made from spring like material that is biased to cause the jaws to either open or close. If desired, forceps 120 may include a plurality of teeth 128 for gripping tissue.

As depicted in FIG. 1(a), a plicator actuator 130 is provided. Actuator 130 is operably coupled to proximal end 112 of plicator 110. Plicator actuator 130 is configured and adapted to adjust the tissue plicator 110 from a first configuration wherein the tissue plicator is disengaged from the target area, wherein jaws 122 are open, to an second configuration wherein jaws 122 of tissue plicator are engaged with the target area. If hinge 126 is a living hinge, actuator can be configured and adapted to oppose the bias of hinge 126. That is, actuator 130 can be adapted to cause jaws 122 to splay apart or come together, as desired.

Actuator can take on a variety of forms. For example, and as depicted in FIG. 1(a), actuator 130 includes a plurality of linkages 132a, 132b operably coupled to a handle 134 having portions 134a and 134b. As portion 134a is moved with respect to 134b, jaws 122 can be caused to move toward or away from one another. The handle 134 can take on a variety of forms. While a two piece push-pull handle 134 is depicted, it is also possible to use other actuators as are known in the art, such as threaded rotating actuators similar to those for retractable sheaths as described in U.S. Pat. No. 6,488,694 to Lau and U.S. Pat. No. 5,906,619 to Olson, the specifications of which are incorporated herein by reference.

Linkages 132a, 132b can take on a variety of forms that permit relative movement. For example, as disclosed in FIG. 1(a), linkages 132a, 132b can be disposed within a sheath 133 that prevents splaying of linkages 132. By way of further example, linkages 132 can be formed concentrically as disclosed in FIG. 2, whereby outer linkage 132a is sleeve shaped and has a distal end 132d that slides along inner linkage 132b over jaws 122 to cause jaws 122 to grip tissue. In addition, other types of actuators are possible, including hydraulically, pneumatically and electromagnetic actuators.

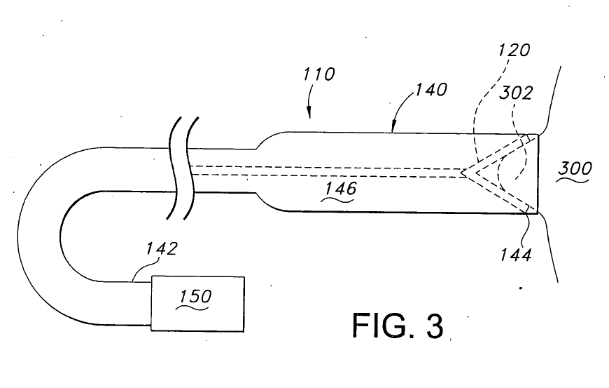

Plicator 110 can grasp tissue 300 to form a plication 302 in a variety of ways. In addition or instead of mechanically grasping the tissue with forceps 120, as depicted in FIG. 3, tissue plicator 110 may also plicate the tissue at least in part by applying suction thereto. In accordance with this aspect, a suction sheath 140 can be provided having a proximal end 142 and a distal end 144 and defining a lumen 146 therethrough. Proximal end 142 of lumen 146 can be placed in fluid communication with a suction source 150. When the suction source 150 is activated, the tissue plicator 110 may plicate the tissue at least in part by drawing the tissue 300 into the lumen under suction from suction source 150. If desired, forceps 120 or similar structure can be disposed within lumen 146 to grasp tissue that has been drawn into lumen 146 under suction. Forceps 120 can initially be provided in a collapsed state when introducing medical device 100 into a patient, and can then expand to cause sheath 140 to expand in a radial direction. This facilitates formation of a larger plication 302 of tissue 300.

Tissue plicator 110 can be made from a variety of materials. Tissue plicator 110 should be made of materials that are sufficiently flexible to traverse the lumenal system of a patient to access the heart. Suitable materials include, for example, surgical grades of stainless steel, nitinol, other alloys, plastic, polymer materials and the like. It is also possible to make at least first and second jaws 122 of forceps 120 at least in part from radiopaque materials that are visible under fluoroscopy, such as platinum gold, barium or iridium, for example. Forceps 120 can also be made from less expensive surgical steel, and plated with radiopaque materials. Similarly, marker bands 121 made from radiopaque material can also be provided as depicted in FIG. 1 (a). By way of further example, materials visible under ultrasound imaging can also be used, such as materials including microparticles, materials having altered surface texture, materials including microbubbles, and the like. Moreover, if magnetic resonance imaging is used, medical device 100 can be formed from materials that are not sensitive to high magnetic fields, such as composite materials including carbon fiber and the like.

In further accordance with the invention, the medical device of the present invention includes a retainer applicator for applying a retainer to the plication to maintain the plication after the medical device is removed from the patient.

For purposes of illustration and not limitation, as embodied herein and as depicted in FIG. 1(a), medical device 100 includes retainer applicator 160. Retainer applicator 160 is preferably operatively associated with the tissue plicator 110, but can be introduced separately, if desired. The retainer applicator 160 is adapted and configured to apply a retainer 200, discussed in detail below, to the plication 302 to maintain the plication 302 after the medical device 100 is removed from the patient.

The retainer applicator 160 can be adapted and configured to deliver the retainer 200 along the tissue plicator 110 to the target region. In accordance with this aspect, the retainer applicator 160 can adapted and configured to deliver the retainer along the outside 115 of the tissue plicator 110 in monorail fashion to the target region. Alternatively, as depicted in FIGS. 1(b)-1(e), the tissue plicator 110 can define a lumen 118 therethrough and the retainer applicator 160 can be adapted and configured to deliver the retainer through the lumen 118 defined by the tissue plicator 110 to the target region T.

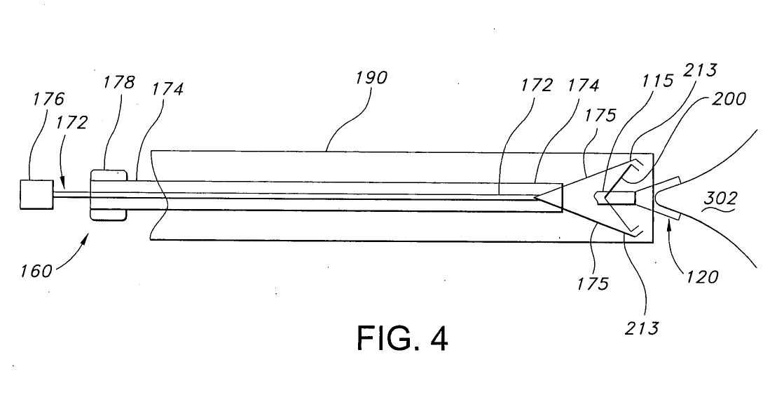

As depicted in FIGS. 1(a) and 4, retainer applicator 160 includes an applicator actuator 170 that can be operably coupled to the retainer applicator 160, wherein the actuator is configured and adapted to affix the retainer 200 onto the plication 302 of tissue 300. As depicted in FIG. 4, applicator actuator 170 includes an advancement mechanism 172 for advancing a retainer to the target region T. Handle 176 can also be provided for actuating the advancement mechanism 172.

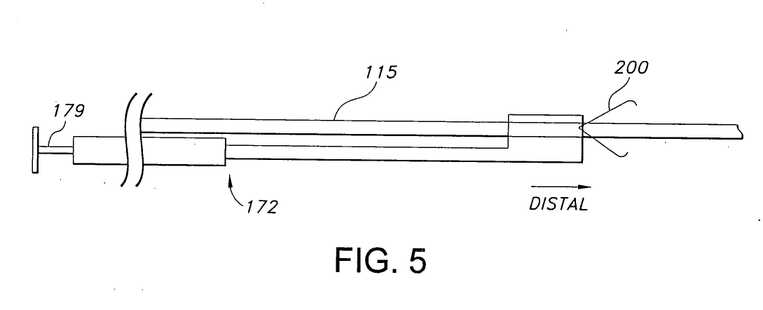

As depicted in FIG. 1(a), advancement mechanism 172 can be provided in the form of a pusher tube that advances retainer 200 along the outside 115 of tissue plicator or through lumen 118 of plicator 110 as depicted in FIG. 1(b). Advancement mechanism 172 could also be provided as a hydraulic piston actuated by a plunger 179 as depicted in FIG. 5 to advance retainer 200 along the outside 115 of plicator 110, among other possible embodiments as disclosed herein. Advancement mechanism 172 could also be a combination of a push-pull arrangement to position the retainer proximate the target area, combined with a threaded fine adjustment to precisely set the retainer over the plication without compromising the tissue by cutting through it with the retainer 200.

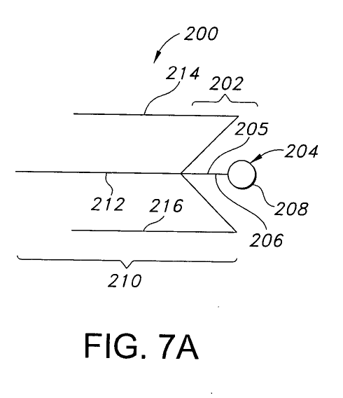

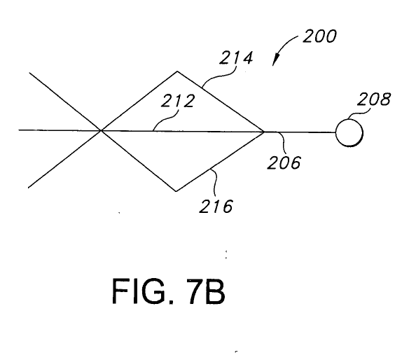

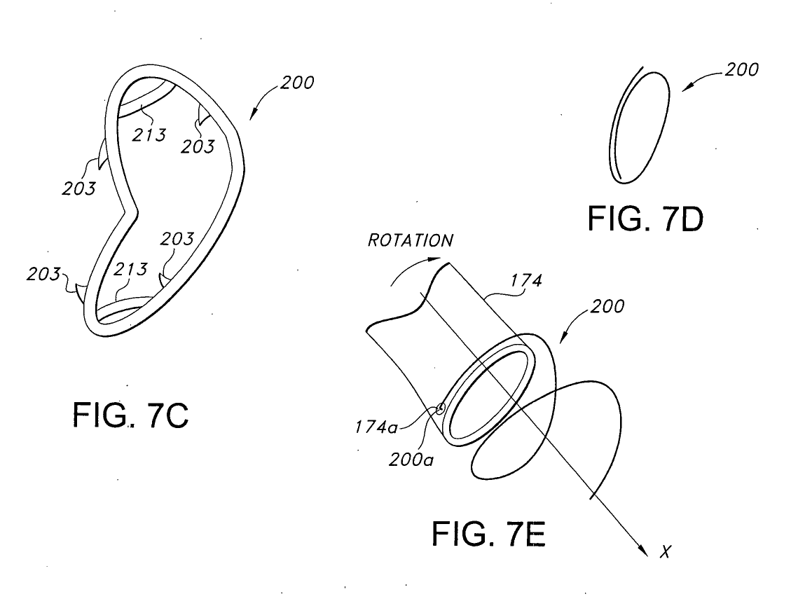

If desired, an engagement mechanism 174 for engaging the retainer 200 with the tissue plication 302 can also be provided. Handle 178 can be provided for actuating the engagement mechanism 174. Engagement mechanism 174 can also take on a variety of forms. For example, and as depicted in FIG. 4, engagement mechanism 174 can include a plurality of jaws 175 for clamping down on retainer 200 to cause it to engage plication 302. Jaws 175 can be actuated by advancing, for example, a tubular member 177 with respect to advancement mechanism over jaws 175 causing them to compress retainer 200 and anchor it into plication 302. By way of further example and as shown in FIG. 7(e), engagement mechanism 174 can be configured to rotate retainer 200 about a longitudinal axis X defined by medical device 100 to affect engagement between retainer 200 and plication 302 by moving retainer 200 through a helical path. Engagement mechanism can be provided in the form of a tubular member that rotates about axis X that is configured to engage helical member 200 in a variety of ways, such as a threaded connection, force-fit, or by having an end 200a of member 200 engage a hole 174a in the periphery of engagement mechanism 174 as depicted in FIG. 7(e).

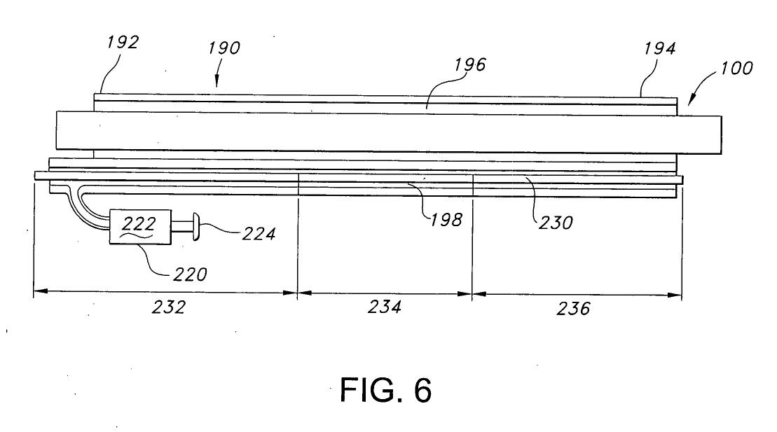

The system described herein also preferably includes an outer catheter 190 (such as a guiding catheter) to facilitate delivery of medical device 100 in combination with retainer 200 to the target region T of a patient. For purposes of illustration only and as depicted in FIG. 6, outer catheter 190 includes a proximal end 192, a distal end 194 and defines a lumen 196 therethrough. Medical device 100 can be disposed within lumen 196 of outer catheter 190 and act as an inner catheter of the system.

Outer catheter 190 can be made from a variety of materials, including multilayer polymeric extrusions, such as those described in U.S. Pat. No. 6,464,683 to Samuelson or U.S. Pat. No. 5,538,510 to Fontirroche, the disclosure of each being incorporated by reference herein in its entirety. Other structures are also possible, including single or multilayer tubes reinforced by braiding, such as metallic braiding material.

As depicted in FIG. 6, outer catheter 190 can further define a second lumen 198 parallel to the first lumen 196. The second lumen 198 can be connected to a source 220 of beneficial agent 222, and the system can be adapted and configured to selectively deliver the beneficial agent 222 to target region T through the second lumen 198 for example, by actuating a plunger 224. The beneficial agent 222 can be chosen from the group consisting of contrast agents, medicaments, viral vectors, and genetic material. Other beneficial agents can also be delivered in this manner, including polymer materials, cells in polymeric matrices, nanoparticles, and the like.

Additionally or alternatively, a stiffening wire 230 can be disposed in the second lumen 198 to impart desired stiffness characteristics to outer catheter 190. The stiffening wire 230 can be movably disposed in the second lumen or can be stationary, if desired. Stiffening wire 230 is provided with a proximal region 232, a medial region 234 and a distal region 236. Stiffening wire 230 can have a varying stiffness along its length. For example, it may be desired to have a stiffening wire with a comparatively stiff proximal region 232 to provide rigidity to the outer catheter 190, and progressively less stiff medial and distal regions 234, 236. Depending on the application at hand, it may be more beneficial to have a stiffening wire with a medial region 234 or distal region 236 that is stiffer than the proximal region 232. Stiffening wire 230 can be made from a variety of materials, including stainless steel, nitinol, various suitable plastics and other alloys. Stiffening wire 230 can also be coated with a lubricious coating to facilitate movement within lumen 198 as described below.

Any surface of various components of the system described herein (e.g., medical device 100, outer catheter 190) or portions thereof can be provided with one or more suitable lubricious coatings to facilitate procedures by reduction of frictional forces. Such coatings can include, for example, hydrophobic materials such as PolyTetraFluoroEthylene (“PTFE”) or silicone oil, or hydrophilic coatings such as Polyvinyl Pyrrolidone (“PVP”). Other coatings are also possible, including, echogenic materials, radiopaque materials and hydrogels, for example.

In another aspect, as disclosed herein, the system of the invention also can include a retainer for maintaining a plication of tissue.

For purposes of illustration, and not limitation, as depicted in FIG. 7(a), retainer 200 is provided. Retainer 200 includes a proximal portion 202 having a proximal end 204, a distal end 206 and a body 205, wherein the proximal end 204 of the main body portion can define a mating portion 208 for mating with the applicator 160. The retainer 200 can be further provided with a distal portion 210 including a first prong 212 adapted and configured to pass through tissue of a patient's vascular system. The retainer can include a second prong 214 attached to the main body portion 202. The second prong 214 can be deformable from an open position for capturing a tissue plication between the first prong and second prong to a closed position for maintaining a tissue plication by the applicator, as depicted in FIG. 7(b). The mating portion 208 of the retainer 200 can define a loop adapted and configured to receive a portion of the applicator as depicted in FIG. 7(a).

Retainer 200 can take on a variety of forms. For example, loop 208 could be omitted and applicator can be configured and adapted to mate with prongs 212 and 214. Loop 208 can also be directly attached to prongs 212, 214 by eliminating body 205. Moreover, the retainer 200 can additional prongs such as third prong 216 attached to the main body portion 200, wherein the second prong 214 and third prong 216 are generally parallel to the main body portion 202 in the open position.

By way of further example and as depicted in FIG. 7(c), retainer 200 can be substantially ring shaped. If desired, the retainer can adapted and configured to be folded about hinge portions 201 by jaws 175 of an applicator 160 about the tissue plication 302 as depicted in FIG. 4. By way of further example, as depicted in FIG. 7d, retainer 200 can be helically shaped and rotated about a longitudinal axis defined by the medical device to introduce the retainer into the target region. Retainer can be provided with one or more barbs 203 to prevent retainer from backing out from tissue 300, as well as one or more tabs 213 to allow for later removal, if desired.

Retainer 200 can be made from a variety of materials, including, for example, shape memory materials, radiopaque materials, resorbable materials, polymeric materials, echogenic materials and/or fluoroscopically visible materials. If made from shape memory material, retainer can be configured to clamp down on plication 302 when it reaches body temperature. For example, the retainer as disclosed in FIG. 7d can be made from shape memory material and trained so that it is an elongate spiral as depicted in 7(e) that compresses longitudinally into a ring shape when its temperature increases as depicted in FIG. 7d.

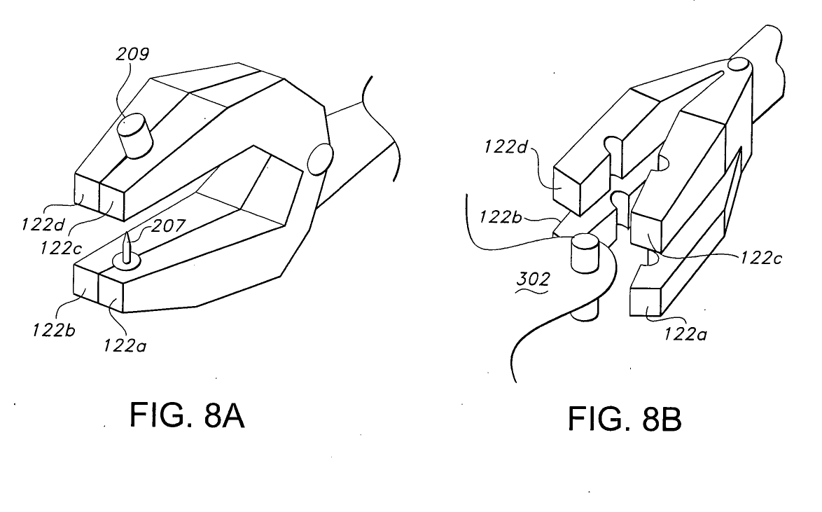

Other variations of the system herein are also possible. For example, a tissue plicator 110 including any desired number of jaws 122 can be used. For example, it is possible to use more than two jaws 122 as disclosed in FIG. 8(a). In the embodiment of FIG. 8(a), four jaws 122 are used to make up forceps 120. Lower jaws 122a and 122b can be moved relative to upper jaws 122c and 122d. In addition, jaws 122a, 122b can be moved laterally with respect to jaws 122c, 122d respectively to facilitate delivery of a retainer 200.

In the embodiment of FIG. 8(a), retainer 200 is initially provided in two separate portions. First portion 207 is trapped between lower jaws 122a, 122b, and second portion 209 is trapped between upper jaws 112c, 122d. Plicator 110 is advanced to a target location T as depicted in FIG. 8(b). The sets of jaws 122 are then brought together to form a plication. As this occurs, first portion 207 and second portion 209 are caused to mate. If the formation of plication 302 has created a beneficial result (such as reduce mitral regurgitation), the lower jaws 122a and 122b can be separated from one another and upper jaws 112c and 122d can be separated from one another to release retainer, and maintain plication 302. Mechanical actuators (not shown) to cause desired movement of jaws 112(a-d) can be designed to create the desired movement.

In accordance with another aspect of the invention, a method of for treating the lumenal system of a patient is provided.

For purposes of illustration and not limitation, as embodied herein, the method includes the steps of providing a inner catheter, such as medical device 100, having a distal portion for creating a plication in tissue such as distal portion 112 of plicator 110.

The method further includes introducing the inner catheter into a lumenal system of a patient, and advancing the distal portion to a target region to be plicated. By way of example, in accordance with one aspect, the method preferably begins with creating an access into the lumenal system of a patient, such as through the femoral artery. A valved adaptor such as a trocar (not shown) is placed into the opening in order to avoid loss of blood. Next, a guidewire 250 can be introduced through the trocar and advanced to the target region T of a patient. The target region can be the mitral valve 310 of a patient, but can be other locations in the lumenal system of the patient, as is desired. The mitral valve 310 can be accessed from the atrial side or the ventricular side, as is desired.

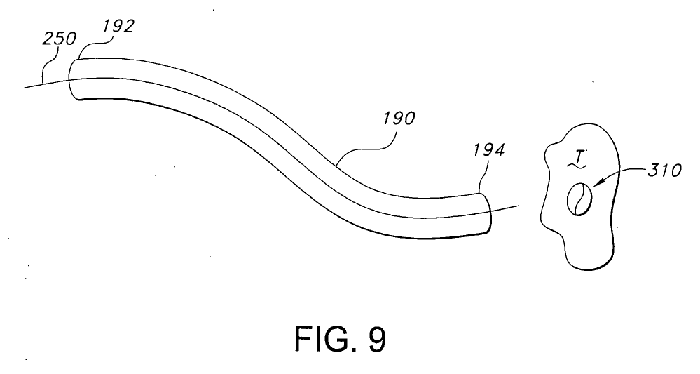

Preferably, as depicted in FIG. 9 and FIGS. 1(b)-1(e), an outer catheter 190 is next introduced into the patient over the guidewire. Distal end 194 of outer catheter 190 is positioned proximate target region T of a patient, such as proximate the mitral valve 310. The procedure is preferably done under visualization of the target region, such as by under fluoroscopy, ultrasound or magnetic resonance imaging.

Next, the guidewire 250 can be withdrawn and medical device 100 is introduced into the lumenal system of a patient through lumen 196 of outer catheter 190 as depicted in FIGS. 1(b)-1(e). Distal end 112 of plicator 110 is moved distally through lumen 196 of outer catheter until jaws 122 are positioned proximate target region T. Jaws 122 are then moved into an open position using plicator actuator 130. Jaws are further advanced against tissue 300 of target region T such that teeth 128, if provided, bite into tissue 300. Actuator 130 is then actuated, causing jaws 122 to close and pull on tissue 300 to form a plication 302, as depicted in FIG. 1(c).

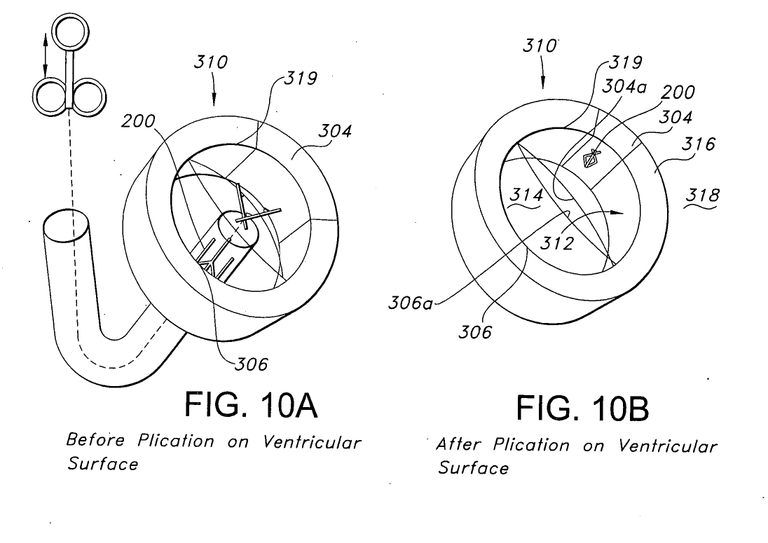

Plication 302 of tissue is preferably formed by pinching tissue along a circumferential direction outside the mitral annulus, proximate the posterior leaflet 304 of mitral valve, as depicted in FIGS. 10(a)-10(b). While plication 302 can be formed in fibrous tissue near the annulus, plication is preferably formed in the muscular tissue of the wall 312 of the ventricle 314 or wall 316 of the atrium 318. The aim of forming plication 302 is to reduce the effective perimeter 320 of mitral valve by pinching it together. If successful, this will ideally cause the edges 304a, 306a of posterior leaflet 304 and anterior leaflet 306 of mitral valve to realign, thereby reducing mitral valve regurgitation. As can be seen, in FIG. 10(a), the perimeter 319 of the mitral valve is reduced as compared to FIG. 10(b), after the procedure.

Once plication 302 is formed, if desired, it is possible to view the effect that formation of plication 302 has had on alignment of leaflets 304, 306. Under fluoroscopy, regurgitation of mitral valve 310 can be viewed during the procedure to determine if forming plication 302 has had a beneficial result. If the result has not been beneficial, plication 302 can be released without permanently altering the tissue. A new plication can then be formed in a different location in an attempt to reduce mitral valve regurgitation. As can be seen, this technique of forming a temporary plication can provide a significant advantage over more invasive procedures since the latter usually require stopping the heart. However, if the result has reduced regurgitation to some extent, the plication 302 can be maintained by applying a retainer 200 to the plication. The retainer 200 can be delivered in any manner, such as described herein.

In certain circumstances, it is also possible to form the plication by using the retainer 200 itself in a single step without first forming a temporary plication. In accordance with this aspect, a medical device 100 is provided having a tissue plicator 110 that is adapted and configured to form a plication of tissue in endocardial muscular tissue proximate the mitral valve of a patient. Tissue plicator can also perform the function of delivering a retainer 200 to a target region T in a single step without forming a plication of tissue 302 prior to delivering retainer 200.

Additional plications 302 proximate the mitral valve 310 radially displaced from one another can be formed, if desired. These plications 302 can be held in place by additional retainers 200 to further reduce the perimeter of the mitral valve 310.

The methods and systems of the present invention, as described above and shown in the drawings, provide for a medical device and method for treating mitral valve regurgitation with superior properties including, for example, greater ease of use and effectiveness. It will be apparent to those skilled in the art that various modifications and variations can be made in the device and method of the present invention without departing from the spirit or scope of the invention. Thus, it is intended that the present invention include modifications and variations that are within the scope of the appended claims and their equivalents.

What is claimed is:

1. A medical device comprising:
a) a tissue plicator adapted and configured to form a plication of tissue proximate a target region of a patient;
b) a retainer applicator operatively associated with the tissue plicator, the retainer applicator adapted and configured to apply a retainer to the plication to maintain the plication after the medical device is removed from the patient.

2. The medical device of claim 1, wherein the tissue plicator plicates tissue by mechanically clamping the tissue.

3. The medical device of claim 2, wherein the tissue plicator includes forceps configured and adapted to mechanically grasp the tissue.

4. The medical device of claim 3, wherein the forceps includes a plurality of teeth for gripping tissue.

5. The medical device of claim 1, further comprising a sheath defining a lumen, wherein the tissue plicator is disposed in the lumen and the tissue plicator plicates the tissue at least in part by applying suction thereto.

6. The medical device of claim 5, wherein the tissue plicator plicates the tissue by drawing the tissue into the lumen.

7. The medical device of claim 6, wherein the tissue plicator is configured and adapted to expand the sheath in a radial direction when grasping tissue.

8. The medical device of claim 1, wherein the tissue plicator is formed at least in part of radiopaque material.

9. The medical device of claim 1, wherein the retainer applicator is adapted and configured to deliver the retainer along the tissue plicator to the target region.

10. The medical device of claim 9, wherein the retainer applicator is adapted and configured to deliver the retainer along the outside of the tissue plicator to the target region.

11. The medical device of claim 9, wherein:
the tissue plicator defines a lumen therethrough; and
the retainer applicator is adapted and configured to deliver the retainer along the lumen defined by the tissue plicator to the target region.

12. The medical device of claim 1, further comprising a plicator actuator operably coupled to the tissue plicator, wherein the plicator actuator is configured and adapted to adjust the tissue plicator from a first configuration wherein the tissue plicator is disengaged from the target area to an second configuration wherein the tissue plicator is engaged with the target area.

13. The medical device of claim 1, further comprising an applicator actuator operably coupled to the retainer applicator, wherein the actuator is configured and adapted to affix the retainer onto the plication of tissue.

14. A medical device comprising:
a tissue plicator adapted and configured to form a plication of tissue in endocardial muscular tissue proximate the mitral valve of a patient.

15. The medical device of claim 14, further comprising:
a retainer applicator operatively associated with the tissue plicator, the retainer applicator adapted and configured to apply a retainer to the plication to maintain the plication after the medical device is removed from the patient.

16. A medical device comprising:
a retainer applicator adapted and configured to apply a retainer to a plication of tissue by rotating the retainer about a longitudinal axis defined by the retainer applicator to maintain the plication after the medical device is removed from the patient.

17. A system comprising:
a) an inner catheter including:
i) a tissue plicator adapted and configured to form a plication of tissue proximate a target region of a patient;
ii) a retainer applicator configured and adapted to apply a retainer to the plication to maintain the plication after the medical device is removed from the patient; and
b) a retainer configured and adapted to maintain the plication of tissue after the device is removed from the patient.

18. The system of claim 17, further comprising an outer catheter defining a first lumen, the inner catheter being disposed in the first lumen.

19. The system of claim 18, wherein the outer catheter defines a second lumen parallel to the first lumen.

20. The system of claim 19, wherein the second lumen is connected to a source of beneficial agent, and the system is adapted and configured to selectively deliver the beneficial agent to the target region.

21. The system of claim 20, wherein the beneficial agent is chosen from the group consisting of contrast agents, medicaments, viral vectors, and genetic material.

22. The system of claim 19, further comprising a movable stiffening wire disposed in the second lumen.

23. The system of claim 22, wherein the stiffening wire has a varying stiffness along its length.

24. The system of claim 17, wherein the retainer includes:
a) a main body portion having a proximal end and a distal end, wherein the proximal end of the main body portion defines a mating portion for mating with the applicator, and a distal end including a first prong adapted and configured to pass through tissue of a patient's vascular system; and
b) a second prong attached to the main body portion, the second prong being deformable from an open position for capturing a tissue plication between the first prong and second prong to a closed position for maintaining a tissue plication by the applicator.

25. The system of claim 24, wherein the mating portion of the retainer defines a loop adapted and configured to receive a portion of the applicator.

26. The system of claim 25, wherein the retainer includes a third prong attached to the main body portion, wherein the second prong and third prong are generally parallel to the main body portion in the open position.

27. The system of claim 17, wherein the retainer is substantially ring shaped.

28. The system of claim 27, wherein the retainer can be deformed from an open position to a closed position for capturing and maintaining a tissue plication.

29. The system of claim 28, wherein the retainer is adapted and configured to be folded by the applicator about the tissue plication.

30. The system of claim 28, wherein the retainer is helically shaped.

31. The system of claim 30, wherein the retainer is rotated about a longitudinal axis defined by the medical device to introduce the retainer into the target region.

32. The system of claim 17, wherein the retainer includes shape memory material.

33. The system of claim 17, wherein the retainer includes radiopaque material.

34. The system of claim 17, wherein the retainer includes resorbable material.

35. The system of claim 17, wherein the retainer includes at least one of a polymeric material, an echogenic material and a fluoroscopically visible material.

36. The system of claim 17, wherein the retainer includes one or more barbs for anchoring the retainer into the patient.

37. A method comprising:
a) providing a inner catheter having a distal portion for creating a plication in tissue;
b) introducing the inner catheter into a lumenal system of a patient, and advancing the distal portion to a target region to be plicated;
c) temporarily plicating tissue proximate the target region to form a first plication;
d) applying a first retainer to the first plication; and
e) removing the inner catheter from the patient.

38. The method of claim 37, wherein the target region is proximate the mitral valve of a patient.

39. The method of claim 38, wherein the first plication is formed on the ventricular wall.

40. The method of claim 38, wherein the first plication is formed on the atrial wall.

41. The method of claim 38, wherein the target region is proximate the posterior leaflet of the mitral valve.

42. The method of claim 37, wherein the retainer is introduced into the patient by sliding it over the inner catheter.

43. The method of claim 38, further comprising the step of observing the circulation through the patient's heart after plicating the tissue but before applying the retainer to determine if mitral regurgitation has been decreased by plicating the tissue.

44. The method of claim 43, wherein the circulation of the patient is observed by using at least one of a fluoroscopic technique and an ultrasonographic technique.

45. The method of claim 37, wherein the inner catheter is introduced into a patient through a lumen of an outer catheter.

46. The method of claim 38, wherein the mitral valve defines a perimeter and the plication is formed to reduce the perimeter of the mitral valve.

47. The method of claim 46, further comprising the steps of:
a) forming second and third plications proximate the mitral valve and radially displaced from the first; and
b) applying second and third retainers to the second and third plications to further reduce the perimeter of the mitral valve.

48. A method comprising:
a) providing a inner catheter having a distal portion for creating a plication in tissue;
b) introducing the inner catheter into a lumenal system of a patient, and advancing the distal portion proximate an endocardial location;
c) engaging endocardial tissue to form a plication therein.

49. The method of claim 48, wherein the endocardial tissue is proximate a mitral valve of a patient.

50. The method of claim 48, wherein the endocardial tissue is muscular tissue.

51. The method of claim 48, wherein the plication is formed temporarily.

52. The method of claim 48, wherein the plication is formed by applying a fastener to the endocardial tissue.

53. The method of claim 48, wherein the plication is formed on the ventricular wall.

54. The method of claim 48, wherein the plication is formed on the atrial wall.

55. The method of claim 48, further comprising applying a retainer to the plication.

56. A method comprising:
a) providing a inner catheter having a distal portion for creating a plication in tissue;
b) introducing the inner catheter into a lumenal system of a patient, and advancing the distal portion proximate an interior surface of the lumenal system;
c) engaging endocardial tissue to form a plication therein; and
d) attaching a retainer to maintain the plication by rotating the retainer about a longitudinal axis defined by the inner catheter.
